SEQUENCE LISTING (2 sequences)

SEQ ID NO: 1 - protein, 4588 aa, Homo sapiens
MGRHLALLLLLLLLFQHFGDSDGSQRLEQTPLQFTHLEYNVTVQENSAAKTYVGHPVKMGVYITHPAWEVRYKIVSGDSENLFKAEEYILGDFCFLRIRTKGGNTAILNREVKDHYTLIVKALEKNTNVEARTKVRVQVLDTNDLRPLFSPTSYSVSLPENTAIRTSIARVSATDADIGTNGEFYYSFKDRTDMFAIHPTSGVIVLTGRLDYLETKLYEMEILAADRGMKLYGSSGISSMAKLTVHIEQANECAPVITAVTLSPSELDRDPAYAIVTVDDCDQGANGDIASLSIVAGDLLQQFRTVRSFPGSKEYKVKAIGGIDWDSHPFGYNLTLQAKDKGTPPQFSSVKVIHVTSPQFKAGPVKFEKDVYRAEISEFAPPNTPVVMVKAIPAYSHLRYVFKSTPGKAKFSLNYNTGLISILEPVKRQQAAHFELEVTTSDRKASTKVLVKVLGANSNPPEFTQTAYKAAFDENVPIGTTVMSLSAVDPDEGENGYVTYSIANLNHVPFAIDHFTGAVSTSENLDYELMPRVYTLRIRASDWGLPYRREVEVLATITLNNLNDNTPLFEKINCEGTIPRDLGVGEQITTVSAIDADELQLVQYQIEAGNELDFFSLNPNSGVLSLKRSLMDGLGAKVSFHSLRITATDGENFATPLYINITVAASHKLVNLQCEETGVAKMLAEKLLQANKLHNQGEVEDIFFDSHSVNAHIPQFRSTLPTGIQVKENQPVGSSVIFMNSTDLDTGFNGKLVYAVSGGNEDSCFMIDMETGMLKILSPLDRETTDKYTLNITVYDLGIPQKAAWRLLHVVVVDANDNPPEFLQESYFVEVSEDKEVHSEIIQVEATDKDLGPNGHVTYSIVTDTDTFSIDSVTGVVNIARPLDRELQHEHSLKIEARDQAREEPQLFSTVVVKVSLEDVNDNPPTFIPPNYRVKVREDLPEGTVIMWLEAHDPDLGQSGQVRYSLLDHGEGNFDVDKLSGAVRIVQQLDFEKKQVYNLTVRAKDKGKPVSLSSTCYVEVEVVDVNENLHPPVFSSFVEKGTVKEDAPVGSLVMTVSAHDEDARRDGEIRYSIRDGSGVGVFKIGEETGVIETSDRLDRESTSHYWLTVFATDQGVVPLSSFIEIYIEVEDVNDNAPQTSEPVYYPEIMENSPKDVSVVQIEAFDPDSSSNDKLMYKITSGNPQGFFSIHPKTGLITTTSRKLDREQQDEHILEVTVTDNGSPPKSTIARVIVKILDENDNKPQFLQKFYKIRLPEREKPDRERNARREPLYHVIATDKDEGPNAEISYSIEDGNEHGKFFIEPKTGVVSSKRFSAAGEYDILSIKAVDNGRPQKSSTTRLHIEWISKPKPSLEPISFEESFFTFTVMESDPVAHMIGVISVEPPGIPLWFDITGGNYDSHFDVDKGTGTIIVAKPLDAEQKSNYNLTVEATDGTTTILTQVFIKVIDTNDHRPQFSTSKYEVVIPEDTAPETEILQISAVDQDEKNKLIYTLQSSRDPLSLKKFRLDPATGSLYTSEKLDHEAVHQHTLTVMVRDQDVPVKRNFARIVVNVSDTNDHAPWFTASSYKGRVYESAAVGSVVLQVTALDKDKGKNAEVLYSIESGNIGNSFMIDPVLGSIKTAKELDRSNQAEYDLMVKATDKGSPPMSEITSVRIFVTIADNASPKFTSKEYSVELSETVSIGSFVGMVTAHSQSSVVYEIKDGNTGDAFDINPHSGTIITQKALDFETLPIYTLIIQGTNMAGLSTNTTVLVHLQDENDNAPVFMQAEYTGLISESASINSVVLTDRNVPLVIRAADADKDSNALLVYHIVEPSVHTYFAIDSSTGAIHTVLSLDYEETSIFHFTVQVHDMGTPRLFAEYAANVTVHVIDINDCPPVFAKPLYEASLLLPTYKGVKVITVNATDADSSAFSQLIYSITEGNIGEKFSMDYKTGALTVQNTTQLRSRYELTVRASDGRFAGLTSVKINVKESKESHLKFTQDVYSAVVKENSTEAETLAVITAIGNPINEPLFYHILNPDRRFKISRTSGVLSTTGTPFDREQQEAFDVVVEVTEEHKPSAVAHVVVKVIVEDQNDNAPVFVNLPYYAVVKVDTEVGHVIRYVTAVDRDSGRNGEVHYYLKEHHEHFQIGPLGEISLKKQFELDTLNKEYLVTVVAKDGGNPAFSAEVIVPITVMNKAMPVFEKPFYSAEIAESIQVHSPVVHVQANSPEGLKVFYSITDGDPFSQFTINFNTGVINVIAPLDFEAHPAYKLSIRATDSLTGAHAEVFVDIIVDDINDNPPVFAQQSYAVTLSEASVIGTSVVQVRATDSDSEPNRGISYQMFGNHSKSHDHFHVDSSTGLISLLRTLDYEQSRQHTIFVRAVDGGMPTLSSDVIVTVDVTDLNDNPPLFEQQIYEARISEHAPHGHFVTCVKAYDADSSDIDKLQYSILSGNDHKHFVIDSATGIITLSNLHRHALKPFYSLNLSVSDGVFRSSTQVHVTVIGGNLHSPAFLQNEYEVELAENAPLHTLVMEVKTTDGDSGIYGHVTYHIVNDFAKDRFYINERGQIFTLEKLDRETPAEKVISVRLMAKDAGGKVAFCTVNVILTDDNDNAPQFRATKYEVNIGSSAAKGTSVVKVLASDADEGSNADITYAIEADSESVKENLEINKLSGVITTKESLIGLENEFFTFFVRAVDNGSPSKESVVLVYVKILPPEMQLPKFSEPFYTFTVSEDVPIGTEIDLIRAEHSGTVLYSLVKGNTPESNRDESFVIDRQSGRLKLEKSLDHETTKWYQFSILARCTQDDHEMVASVDVSIQVKDANDNSPVFESSPYEAFIVENLPGGSRVIQIRASDADSGTNGQVMYSLDQSQSVEVIESFAINMETGWITTLKELDHEKRDNYQIKVVASDHGEKIQLSSTAIVDVTVTDVNDSPPRFTAEIYKGTVSEDDPQGGVIAILSTTDADSEEINRQVTYFITGGDPLGQFAVETIQNEWKVYVKKPLDREKRDNYLLTITATDGTFSSKAIVEVKVLDANDNSPVCEKTLYSDTIPEDVLPGKLIMQISATDADIRSNAEITYTLLGSGAEKFKLNPDTGELKTSTPLDREEQAVYHLLVRATDGGGRFCQASIVLTLEDVNDNAPEFSADPYAITVFENTEPGTLLTRVQATDADAGLNRKILYSLIDSADGQFSINELSGIIQLEKPLDRELQAVYTLSLKAVDQGLPRRLTATGTVIVSVLDINDNPPVFEYREYGATVSEDILVGTEVLQVYAASRDIEANAEITYSIISGNEHGKFSIDSKTGAVFIIENLDYESSHEYYLTVEATDGGTPSLSDVATVNVNVTDINDNTPVFSQDTYTTVISEDAVLEQSVITVMADDADGPSNSHIHYSIIDGNQGSSFTIDPVRGEVKVTKLLDRETISGYTLTVQASDNGSPPRVNTTTVNIDVSDVNDNAPVFSRGNYSVIIQENKPVGFSVLQLVVTDEDSSHNGPPFFFTIVTGNDEKAFEVNPQGVLLTSSAIKRKEKDHYLLQVKVADNGKPQLSSLTYIDIRVIEESIYPPAILPLEIFITSSGEEYSGGVIGKIHATDQDVYDTLTYSLDPQMDNLFSVSSTGGKLIAHKKLDIGQYLLNVSVTDGKFTTVADITVHIRQVTQEMLNHTIAIRFANLTPEEFVGDYWRNFQRALRNILGVRRNDIQIVSLQSSEPHPHLDVLLFVEKPGSAQISTKQLLHKINSSVTDIEEIIGVRILNVFQKLCAGLDCPWKFCDEKVSVDESVMSTHSTARLSFVTPRHHRAAVCLCKEGRCPPVHHGCEDDPCPEGSECVSDPWEEKHTCVCPSGRFGQCPGSSSMTLTGNSYVKYRLTENENKLEMKLTMRLRTYSTHAVVMYARGTDYSILEIHHGRLQYKFDCGSGPGIVSVQSIQVNDGQWHAVALEVNGNYARLVLDQVHTASGTAPGTLKTLNLDNYVFFGGHIRQQGTRHGRSPQVGNGFRGCMDSIYLNGQELPLNSKPRSYAHIEESVDVSPGCFLTATEDCASNPCQNGGVCNPSPAGGYYCKCSALYIGTHCEISVNPCSSKPCLYGGTCVVDNGGFVCQCRGLYTGQRCQLSPYCKDEPCKNGGTCFDSLDGAVCQCDSGFRGERCQSDIDECSGNPCLHGALCENTHGSYHCNCSHEYRGRHCEDAAPNQYVSTPWNIGLAEGIGIVVFVAGIFLLVVVFVLCRKMISRKKKHQAEPKDKHLGPATAFLQRPYFDSKLNKNIYSDIPPQVPVRPISYTPSIPSDSRNNLDRNSFEGSAIPEHPEFSTFNPESVHGHRKAVAVCSVAPNLPPPPPSNSPSDSDSIQKPSWDFDYDTKVVDLDPCLSKKPLEEKPSQPYSARESLSEVQSLSSFQSESCDDNGYHWDTSDWMPSVPLPDIQEFPNYEVIDEQTPLYSADPNAIDTDYYPGGYDIESDFPPPPEDFPAADELPPLPPEFSNQFESIHPPRDMPAAGSLGSSSRNRQRFNLNQYLPNFYPLDMSEPQTKGTGENSTCREPHAPYPPGYQRHFEAPAVESMPMSVYASTASCSDVSACCEVESEVMMSDYESGDDGHFEEVTIPPLDSQQHTEV

SEQ ID NO: 2 - protein, 13 aa, synthetic construct
FSAAGEYDILSIK

(19) United States
(12) Patent Application Publication
(10) Pub. No.: US 2025/0189523 A1
(43) Pub. Date: Jun. 12, 2025

(54) METHODS AND MATERIALS FOR IDENTIFYING AND TREATING MEMBRANOUS NEPHROPATHY

(71) Applicant: Mayo Foundation for Medical Education and Research, Rochester, MN (US)

(72) Inventors: Sanjeev Sethi, Rochester, MN (US); Fernando C. Fervenza, Rochester, MN (US); Benjamin J. Madden, Stewartville, MN (US)

(21) Appl. No.: 18/843,878

(22) PCT Filed: Mar. 3, 2023
(86) PCT No.: PCT/US2023/063654

§ 371 Date: Sep. 4, 2024

Related U.S. Application Data
(60) Provisional application No. 63/318,171, filed on Mar. 9, 2022.

(51) Int. Cl. G01N 33/564; A61K 31/196; A61K 31/436; A61K 38/13; C07K 16/28; G01N 33/68

(52) U.S. Cl. CPC G01N 33/564; A61K 31/196; A61K 31/436; A61K 38/13; C07K 16/2887; G01N 33/6893; G01N 2800/24; G01N 2800/347

(57) ABSTRACT

This document relates to methods and materials involved in identifying and/or treating mammals having membranous nephropathy (e.g., membranous nephropathy with an elevated level of a protocadherin FAT1 (FAT1) polypeptide in the glomerular basement membrane (GBM)). For example, methods and materials for administering one or more immunosuppressive agents to treat a mammal (e.g., a human) having membranous nephropathy are provided.

### CROSS-REFERENCE TO RELATED APPLICATIONS

This application claims the benefit of U.S. Patent Application Ser. No. 63/318,171, filed on Mar. 9, 2022. The disclosure of the prior application is considered part of (and is incorporated by reference in) the disclosure of this application.

### SEQUENCE LISTING

This application contains a Sequence Listing that has been submitted electronically as an XML file named “07039-2120WO1.xml.” The XML file, created on Feb. 23, 2023, is 7030 bytes in size. The material in the XML file is hereby incorporated by reference in its entirety.

### TECHNICAL FIELD

This document relates to methods and materials involved in identifying and/or treating mammals having membranous nephropathy (e.g., membranous nephropathy with an elevated level of a protocadherin FAT1 (FAT1) polypeptide in the glomerular basement membrane (GBM)). For example, this document provides methods and materials for administering one or more immunosuppressive agents to treat a mammal (e.g., a human) having membranous nephropathy.

### BACKGROUND INFORMATION

Membranous nephropathy results from subepithelial deposition of immune complexes along the GBM. Membranous nephropathy is often classified into primary membranous nephropathy, where there is no identifiable underlying disease association, and secondary membranous nephropathy, where membranous nephropathy may be associated with an autoimmune disease, infection, malignancy, hematopoietic stem cell transplant, etc. (Beck et al., J. Clin. Invest., 124:2307-2314 (2014); Ronco et al., The Lancet, 385:1983-1992 (2015); and Couser, Clin. J. Am. Soc. Nephrol., 12:983-997 (2017)). Target antigens for many types of membranous nephropathy have been identified (Beck et al., New Eng. J. Med., 361:11-21 (2009); Tomas et al., New Eng. J. Med, 371:2277-2287 (2014); Sethi, J. Am. Soc. Nephrol., 32:268 (2021); Sethi et al., J. Am. Soc. Nephrol, 30:1123-1136 (2019); Sethi et al., Kidney Int., 97:163-174 (2020); Sethi et al., Kidney Int., 98:1253-1265 (2020); Sethi et al., J. Am. Soc. Nephrol., 32:1249-1261 (2021); Caza et al., Kidney Int., 100:171-181 (2020); and Al-Rabadi et al., J. Am. Soc. Nephrol., 32:1666-1681 (2021)).

Hematopoietic stem cell transplant (HSCT) can cause graft versus host disease (GVHD), and a complication of chronic GVHD is membranous nephropathy developing in the setting of HSCT (Troxell et al., Adv. Anat. Pathol., 21:330-340 (2014); and Chang et al., Clin. J. Am. Soc. Nephrol., 2:1014-1023 (2007)). The target antigen(s) in HSCT-associated membranous nephropathy remain elusive.

### SUMMARY

This document provides methods and materials involved in identifying and/or treating mammals (e.g., humans) having membranous nephropathy (e.g., membranous nephropathy with an elevated level of a FAT1 polypeptide in the GBM). For example, this document provides methods and materials for identifying a mammal (e.g., a human) having membranous nephropathy as having an elevated level of a FAT1 polypeptide in the GBM that can serve as a target antigen in membranous nephropathy. This document also provides methods and materials for identifying a mammal (e.g., a human) having membranous nephropathy as having the presence of autoantibodies having binding specificity for a FAT1 polypeptide. As described herein, mammals (e.g., humans) having membranous nephropathy can be identified as having an elevated level of a FAT1 polypeptide in the GBM. In such cases, the mammal can be classified as having a form of membranous nephropathy that includes an elevated level of a FAT1 polypeptide in the GBM. As also described herein, mammals (e.g., humans) having membranous nephropathy can be identified as having autoantibodies having binding specificity for a FAT1 polypeptide. In such cases, the mammal can be classified as having a form of membranous nephropathy that includes the presence of autoantibodies having binding specificity for a FAT1 polypeptide. Identifying mammals (e.g., humans) as having membranous nephropathy that includes an elevated level of a FAT1 polypeptide in the GBM and/or that includes the presence of autoantibodies having binding specificity for a FAT1 polypeptide can allow clinicians and patients to proceed with appropriate membranous nephropathy treatment options.

This document also provides methods and materials for treating membranous nephropathy. For example, a mammal (e.g., a human) having membranous nephropathy that was identified as having an elevated level of a FAT1 polypeptide in the GBM, as having autoantibodies having binding specificity for a FAT1 polypeptide, or as having both an elevated level of a FAT1 polypeptide in the GBM and autoantibodies having binding specificity for a FAT1 polypeptide can be administered one or more immunosuppressive agents (e.g., corticosteroids, cyclosporine, or a B-cell reduction or depletion agent such as Rituximab) to reduce inflammation and/or B-cell autoantibody production. As described herein, mammals (e.g., humans) having membranous nephropathy and identified as having an elevated level of a FAT1 polypeptide in the GBM and/or as having autoantibodies having binding specificity for a FAT1 polypeptide have a form of membranous nephropathy that is caused by the presence of antigen-autoantibody complexes where the antigen is a FAT1 polypeptide. In such cases, the mammal (e.g., human) can be effectively treated using one or more immunosuppressive agents (e.g., corticosteroids, cyclosporine, or a B-cell reduction or depletion agent such as Rituximab) to reduce inflammation and/or B-cell autoantibody production. Having the ability to administer one or more immunosuppressive agents to mammals (e.g., humans) (a) having membranous nephropathy and (b) identified as having an elevated level of a FAT1 polypeptide in the GBM and/or as having autoantibodies having binding specificity for a FAT1 polypeptide can allow clinicians and patients to treat membranous nephropathy effectively.

In some cases, identification of the target antigen and autoantibodies can be involved in the diagnosis and/or management of a mammal (e.g., a human) with membranous nephropathy. For example, a mammal (e.g., a human) having membranous nephropathy (e.g., membranous nephropathy having an elevated level of a FAT1 polypeptide in the GBM and/or having autoantibodies having binding specificity for a FAT1 polypeptide) can be administered one or more immunosuppressive agents (e.g., corticosteroids, cyclosporine, or a B-cell reduction or depletion agent such as Rituximab) to treat membranous nephropathy. In some cases, the response to the immunosuppressive treatment can be monitored for a decrease or complete elimination of the autoantibodies having binding specificity for a FAT1 polypeptide. In some cases, the response to treatment can be monitored by examining a kidney biopsy for a decrease or elimination of a FAT1 polypeptide). In some cases, a mammal (e.g., a human) having membranous nephropathy can be administered one or more immunosuppressive agents (e.g., corticosteroids, cyclosporine, or a B-cell reduction or depletion agent such as Rituximab) to treat membranous nephropathy based on the presence of an autoantibody having binding specificity for a FAT1 polypeptide in the absence of evaluating a kidney biopsy for an elevated level of a FAT1 polypeptide. Although kidney biopsies showing an accumulation of a FAT1 polypeptide in GBM may be considered an effective manner for diagnosis of membranous nephropathy, the presence of autoantibodies having binding specificity for a FAT1 polypeptide can be used to identify membranous nephropathy associated with accumulation of FAT1 polypeptides without the need for a kidney biopsy.

In general, one aspect of this document features methods for identifying a mammal comprising an hematopoietic stem cell transplant as having membranous nephropathy comprising an elevated level of a FAT1 polypeptide within kidney tissue of the mammal. The methods can include, or consist essentially of, (a) determining the presence or absence of autoantibodies specific for a FAT1 polypeptide within a sample obtained from a mammal, (b) classifying the mammal as having a membranous nephropathy if the autoantibodies are present within the mammal, and (c) classifying the mammal as not having the membranous nephropathy if the autoantibodies are absent within the mammal. The mammal can be a human. The sample can be a blood sample. The membranous nephropathy can lack an elevated level of a protocadherin-7 (PCDH7) polypeptide, a semaphorin-3B (SEMA3B) polypeptide, a neural epidermal growth factor (EGF)-like 1 (NELL-1) polypeptide, an exostosin 1 (EXT1) polypeptide, an exostosin 2 (EXT2) polypeptide, a PLA2R polypeptide, or a THSD7A polypeptide within the kidney tissue. The membranous nephropathy can lack an elevated level of a PCDH7 polypeptide, a SEMA3B polypeptide, a NELL-1 polypeptide, an EXT1 polypeptide, an EXT2 polypeptide, a PLA2R polypeptide, and a THSD7A polypeptide within the kidney tissue. The method can include detecting the presence of the autoantibodies and classifying the mammal as having the membranous nephropathy. The method can include detecting the absence of the autoantibodies and classifying the mammal as not having the membranous nephropathy.

In another aspect, this document features methods for identifying a mammal comprising an hematopoietic stem cell transplant as having membranous nephropathy having kidney tissue comprising an elevated level of a FAT1 polypeptide. The methods can include, or consist essentially of, (a) determining the presence or absence of a kidney tissue comprising an elevated level of a FAT1 polypeptide within a sample obtained from a mammal, (b) classifying the mammal as having the membranous nephropathy if the presence is determined, and (c) classifying the mammal as not having the membranous nephropathy if the absence is determined. The mammal can be a human. The kidney tissue can lack an elevated level of a PCDH7 polypeptide, a SEMA3B polypeptide, a NELL-1 polypeptide, an EXT1 polypeptide, an EXT2 polypeptide, a PLA2R polypeptide, or a THSD7A polypeptide. The kidney tissue can lack an elevated level of a PCDH7 polypeptide, a SEMA3B polypeptide, a NELL-1 polypeptide, an EXT1 polypeptide, an EXT2 polypeptide, a PLA2R polypeptide, and a THSD7A polypeptide. The method can include detecting the presence and classifying the mammal as having the membranous nephropathy. The method can include detecting the absence and classifying the mammal as not having the membranous nephropathy.

In another aspect, this document features methods for identifying a mammal comprising an hematopoietic stem cell transplant as having membranous nephropathy having autoantibodies specific for a FAT1 polypeptide. The methods can include, or consist essentially of, (a) determining the presence or absence of autoantibodies specific for a FAT1 polypeptide within a sample obtained from a mammal, (b) classifying the mammal as having the membranous nephropathy if the autoantibodies are present within the mammal, and (c) classifying the mammal as not having the membranous nephropathy if the autoantibodies are absent within the mammal. The mammal can be a human. The sample can be a blood sample. The kidney tissue of the mammal can lack an elevated level of a PCDH7 polypeptide, a SEMA3B polypeptide, a NELL-1 polypeptide, an EXT1 polypeptide, an EXT2 polypeptide, a PLA2R polypeptide, or a THSD7A polypeptide. The kidney tissue of the mammal can lack an elevated level of a PCDH7 polypeptide, a SEMA3B polypeptide, a NELL-1 polypeptide, an EXT1 polypeptide, an EXT2 polypeptide, a PLA2R polypeptide, and a THSD7A polypeptide. The method can include detecting the presence and classifying the mammal as having said membranous nephropathy. The method can include detecting the absence and classifying the mammal as not having the membranous nephropathy.

In another aspect, this document features methods for treating a mammal having membranous nephropathy. The methods can include, or consist essentially of, (a) identifying a mammal as having membranous nephropathy comprising (i) autoantibodies specific for a FAT1 polypeptide or (ii) kidney tissue comprising an elevated level of the FAT1 polypeptide, and (b) administering an immunosuppressant to the mammal. The mammal can be a human. The mammal can be identified as having the membranous nephropathy comprising the autoantibodies. The mammal can be identified as having the membranous nephropathy comprising the kidney tissue. The immunosuppressant can be a B-cell inhibitor. The B-cell inhibitor can be rituximab. The immunosuppressant can be a calcineurin inhibitor. The calcineurin inhibitor can be cyclosporine or tacrolimus. The immunosuppressant can be an mTOR inhibitor. The mTOR inhibitor can be sirolimus or everolimus. The immunosuppressant can be a DNA damage inducer. The DNA damage inducer can be chlorambucil. The level of autoantibodies present within the mammal can be reduced by at least 5 percent following the administering step. The level of autoantibodies present within the mammal can be reduced by at least 25 percent following the administering step. The level of autoantibodies present within the mammal can be reduced by at least 50 percent following the administering step.

In another aspect, this document features methods for treating a mammal having membranous nephropathy. The methods can include, or consist essentially of, administering an immunosuppressant to a mammal identified as having membranous nephropathy comprising (i) autoantibodies specific for a FAT1 polypeptide or (ii) kidney tissue comprising an elevated level of the FAT1 polypeptide. The mammal can be a human. The mammal can have been identified as having the membranous nephropathy comprising the autoantibodies. The mammal can have been identified as having the membranous nephropathy comprising the kidney tissue. The immunosuppressant can be a B-cell inhibitor. The B-cell inhibitor can be rituximab. The immunosuppressant can be a calcineurin inhibitor. The calcineurin inhibitor can be cyclosporine or tacrolimus. The immunosuppressant can be an mTOR inhibitor. The mTOR inhibitor can be sirolimus or everolimus. The immunosuppressant can be a DNA damage inducer. The DNA damage inducer can be chlorambucil. The level of autoantibodies present within the mammal can be reduced by at least 5 percent following the administering step. The level of autoantibodies present within the mammal can be reduced by at least 25 percent following the administering step. The level of autoantibodies present within the mammal can be reduced by at least 50 percent following the administering step.

In another aspect, this document features methods for treating a mammal having membranous nephropathy and kidney tissue comprising an elevated level of a FAT1 polypeptide. The methods can include, or consist essentially of, administering an immunosuppressant to a mammal having membranous nephropathy and kidney tissue comprising an elevated level of a FAT1 polypeptide. The mammal can be a human. The mammal can have autoantibodies specific for the polypeptide. The mammal can have been identified as having the kidney tissue. The immunosuppressant can be a B-cell inhibitor. The B-cell inhibitor can be rituximab. The immunosuppressant can be a calcineurin inhibitor. The calcineurin inhibitor can be cyclosporine or tacrolimus. The immunosuppressant can be an mTOR inhibitor. The mTOR inhibitor can be sirolimus or everolimus. The immunosuppressant can be a DNA damage inducer. The DNA damage inducer can be chlorambucil. The level of autoantibodies present within the mammal can be reduced by at least 5 percent following the administering step. The level of autoantibodies present within the mammal can be reduced by at least 25 percent following the administering step. The level of autoantibodies present within the mammal can be reduced by at least 50 percent following the administering step.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Discovery and validation cohorts of FAT1-associated membranous nephropathy. In the discovery cohort (Mayo Clinic cohort, cases 1-5, and cases 11-14), MS/MS was performed in 250 cases to look for novel polypeptides in PLA2R-negative membranous nephropathy. Six cases were positive for a unique FAT1 polypeptide. All cases had history of HSCT. Immunohistochemistry (IHC) was performed on 4 of the 6 cases and showed granular GBM staining for the FAT1 polypeptide. In the validation cohorts (Cedar Sinai cohort, case 6-10), MS/MS was performed in 5 known cases of HSCT-associated membranous nephropathy. All cases were PLA2R-negative. All 5 cases were positive for a unique FAT1 polypeptide by MS/MS. IHC was performed on all 5 cases and showed granular GBM staining for FAT1 polypeptide.

FIGS. 2A-2C. Proteomic Identification of FAT1 polypeptides in HSCT-associated PLA2R-negative membranous nephropathy (MN). Glomeruli were microdissected and analyzed using mass spectrometry. FIG. 2A shows detection of FAT1 in 11 cases of PLA2R-negative membranous nephropathy (top row). Numbers in shaded boxes represent total spectral counts of MS/MS matches to a respective polypeptide. All 11 cases show moderate total spectral counts for FAT1 polypeptides and immunoglobulins, baseline spectral counts of PLA2R were detected in 4 of 10 cases. For comparison, the pooled total spectral counts from 6 control cases (time 0 protocol transplant biopsies) are also shown. FAT1 polypeptide was not present in the control cases. FIG. 2B shows a representative sequence coverage map of a FAT1 polypeptide (SEQ ID NO:1) from one case. Amino acids highlighted and in bold letters are the amino acids detected. FIG. 2C shows an example of MS/MS spectra match to a sequence from the FAT1 polypeptide. Example MS/MS spectra of ion 707.36[M+2H] 2+ matched to the FAT1 polypeptide sequence FSAAGEYDILSIK (SEQ ID NO:2).

FIGS. 3A-3C. IHC and confocal immunofluorescence microscopy for FAT1 polypeptides in HSCT-associated membranous nephropathy (MN) and control cases. FIG. 3 A shows that IHC for FAT1 polypeptide is positive in FAT1-associated membranous nephropathy. Nine cases show bright granular capillary wall staining for FAT1 polypeptides along the glomerular basement membranes. Each case number of FAT1-associated membranous nephropathy is shown. Note positive FAT1 polypeptide staining in tubular epithelial cells. Protease treatment was required for IHC staining. Two cases without protease treatment are negative for FAT1 polypeptides (all 40×). In two cases, tissue was not available for IHC. FIG. 3B shows a confocal immunofluorescence microscopy analysis: Detection of FAT1 polypeptides in glomerular immune deposits. Glomeruli are positive for anti-FAT1 antibodies (top panel) along the capillary walls in two cases of HSCT-associated membranous nephropathy (one each from discovery cohort and validation cohort) and negative for anti-FAT1 antibodies in two cases of PLA2R-associated membranous nephropathy. FIG. 3C shows control cases. IHC of control cases shows no glomerular capillary wall FAT1 polypeptide staining after protease treatment in a normal kidney, minimal change disease, PLA2R-associated membranous nephropathy, and a time 0 transplant biopsy. Note the positive FAT1 polypeptide staining of the tubules (all 40×).

FIGS. 4A-4B. Western Blot analysis showing IgG from eluate and from serum of an index case binds to reduced FAT1 polypeptides (400 ng loaded in each lane). FIG. 4A shows a representative control: Reduced FAT1 polypeptides are detected by rabbit anti-human FAT1 antibodies (0.2 μg/mL) at approximately 33 kDa (arrow) (lane 1). Eluate: reduced FAT1 polypeptides are detected using eluate from FAT-1-associated membranous nephropathy using a secondary anti-human IgG (1:5000) (lane 2) and more specifically using a secondary anti-human IgG4 (1:500) (lane 4). The binding is not detected using eluate from PLA2R-associated membranous nephropathy (lane 3 and lane 5). Serum: reduced FAT1 polypeptides were also detected using index patient serum (1:100 and 1:50) and detected using anti-human IgG4 (1:500) (lane 6) and using anti-human IgG (1:5000) (lane 7). FIG. 4B shows that reduced FAT1 polypeptides are detected by anti-human IgG4 (1:500) in serum from an index case (1:100 dilution) (lane 1) but not in serum from patients with PLA2R associated-membranous nephropathy (lane 2), PLA2R negative membranous nephropathy (lane 3), IgA nephropathy (lane 4), MPO-AAV (lane 5), or in serum from a healthy control (lane 6).

FIG. 5. Biopsy finding of a representative case (patient 1) of FAT1-associated membranous nephropathy. Light microscopy (panel A) showing focally thickened glomerular basement membranes (periodic acid Schiff stain 60×). Immunofluorescence microscopy (panels B and C) showing bright 3+ capillary wall staining for IgG (panel B) and IgG4 along the capillary walls (panel C). Electron microscopy (panel D) showing subepithelial electron dense deposits (7140×). Granular capillary wall staining for FAT1 polypeptides (panels E and F).

### DETAILED DESCRIPTION

This document provides methods and materials for identifying and/or treating mammals (e.g., humans) having membranous nephropathy (e.g., membranous nephropathy with an elevated level of a FAT1 polypeptide in the GBM). For example, this document provides methods and materials for identifying a mammal (e.g., a human) having membranous nephropathy as having (a) autoantibodies specific for a FAT1 polypeptide and/or (b) a GBM having an elevated level of a FAT1 polypeptide.

Any appropriate mammal having membranous nephropathy can be identified as having (a) autoantibodies specific for a FAT1 polypeptide and/or (b) kidney tissue (e.g., GBM) having an elevated level of a FAT1 polypeptide. In some cases, a mammal having membranous nephropathy also can have received a transplant. For example, a mammal having membranous nephropathy also can be a mammal that received a hematopoietic stem cell transplant (HSCT). For example, a mammal having membranous nephropathy also can have received a kidney transplant. Examples of mammals having membranous nephropathy that can be identified as having (a) autoantibodies specific for a FAT1 polypeptide and/or (b) kidney tissue (e.g., GBM) having an elevated level of a FAT1 polypeptide as described herein include, without limitation, primates (e.g., humans and monkeys), dogs, cats, horses, cows, pigs, sheep, rabbits, mice, and rats. For example, humans having membranous nephropathy can be identified as having (a) autoantibodies specific for a FAT1 polypeptide and/or (b) kidney tissue such as GBM having an elevated level of a FAT1 polypeptide as described herein.

Any appropriate method can be used to determine if a mammal (e.g., a human) has autoantibodies specific for a FAT1 polypeptide. For example, immunological assays using a FAT1 polypeptide (or a fragment thereof capable of binding to an anti-FAT1 antibody) can be used to determine if a sample contains autoantibodies specific for a FAT1 polypeptide. In some cases, an immobilized FAT1 polypeptide (or an immobilized fragment thereof) can be used to capture an anti-FAT1 autoantibody if present within a sample being tested, and an anti-Ig antibody (e.g., an anti-human IgG antibody when testing for human autoantibodies) can be used to determine whether or not autoantibodies were captured. In some cases, an anti-Ig antibody can be labeled (e.g., fluorescently or enzymatically labeled) to aid in detection. Any appropriate sample can be used to determine if a mammal (e.g., a human) has autoantibodies specific for a FAT1 polypeptide. For example, blood samples (e.g., whole blood samples, serum samples, and plasma samples) or urine samples obtained from a mammal being tested can be used to determine if a mammal (e.g., a human) has autoantibodies specific for a FAT1 polypeptide.

Any appropriate method can be used to determine if a mammal (e.g., a human) has kidney tissue (e.g., GBM) having an elevated level of a FAT1 polypeptide. For example, immunological techniques such as IHC techniques, immunofluorescence (IF) techniques, or western blot techniques can be used to determine if a mammal (e.g., a human) has kidney tissue (e.g., GBM) having an elevated level of a FAT1 polypeptide. In some cases, a kidney tissue sample obtained from a mammal to be tested can be stained using an anti-FAT1 antibody to determine if the mammal has kidney tissue (e.g., GBM) having an elevated level of FAT1 polypeptides. Any appropriate sample can be used to determine if a mammal (e.g., a human) has kidney tissue (e.g., GBM) having an elevated level of a FAT1 polypeptide. For example, kidney tissue biopsies can be obtained from a mammal (e.g., a human) being tested and used to determine if the mammal (e.g., the human) has kidney tissue (e.g., GBM) having an elevated level of a FAT1 polypeptide.

The term “elevated level” as used herein with respect to a FAT1 polypeptide level refers to a level of FAT1 polypeptides present within kidney tissue (e.g., GBM) that is greater (e.g., at least 10, 25, 35, 45, 50, 55, 65, 75, 80, 90, or 100 percent greater) than the median level of FAT1 polypeptides present within normal kidney tissue (e.g., a normal GBM) of comparable mammals not having membranous nephropathy.

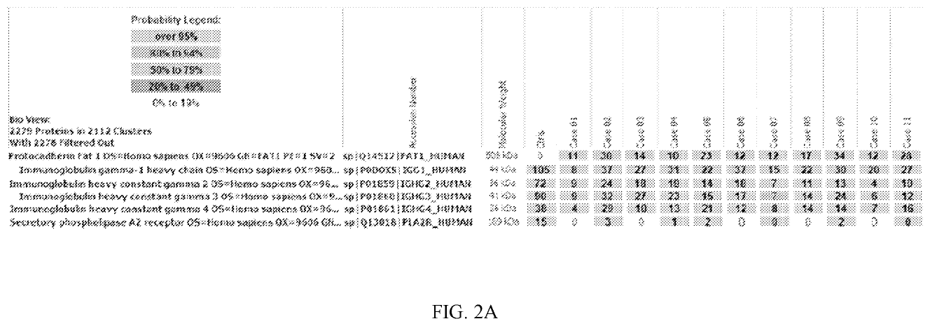

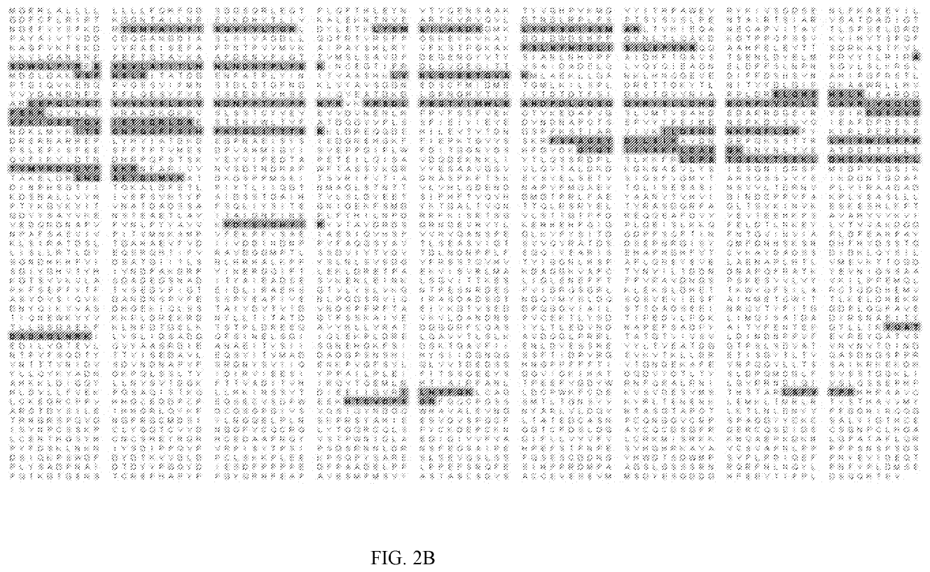

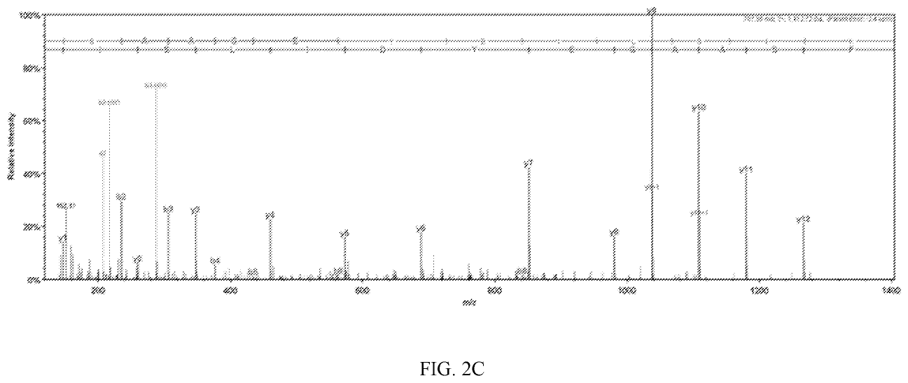

A FAT1 polypeptide can include any appropriate amino acid sequence. Examples of human FAT1 polypeptides include, without limitation, those polypeptides having the amino acid sequence set forth in SEQ ID NO:1 (see, e.g., FIG. 2B) and polypeptides having the amino acid sequence set forth in Uniprot accession Q14517. In some cases, the amino acid sequence of a FAT1 polypeptide can have a sequence that deviates from the nucleotide sequence set forth in SEQ ID NO:1, sometimes referred to as a variant sequence. For example, a FAT1 polypeptide can have an amino acid sequence that includes one or more modifications (e.g., deletions, insertions, and substitutions) to the amino acid sequence set forth in SEQ ID NO:1. For example, an amino acid sequence of a FAT1 polypeptide can have at least 80% sequence identity (e.g., about 82% sequence identity, about 85% sequence identity, about 88% sequence identity, about 90% sequence identity, about 93% sequence identity, about 95% sequence identity, about 97% sequence identity, about 98% sequence identity, or about 99% sequence identity) to the amino acid sequence set forth in SEQ ID NO: 1.

Percent sequence identity is calculated by determining the number of matched positions in aligned amino acid sequences, dividing the number of matched positions by the total number of aligned amino acids, and multiplying by 100. A matched position refers to a position in which identical amino acid residues occur at the same position in aligned sequences. Sequences can be aligned using the algorithm described by Altschul et al. (Nucleic Acids Res., 25:3389-3402 (1997)) as incorporated into BLAST (basic local alignment search tool) programs, available at ncbi.nlm.nih.gov on the World Wide Web. BLAST searches or alignments can be performed to determine percent sequence identity between an amino acid and any other sequence or portion thereof using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between an amino acid sequence and another sequence, the default parameters of the respective programs can be used. In some cases, a human FAT1 polypeptide can have the amino acid sequence set forth in FIG. 2B.

Once a mammal (e.g., a human) having membranous nephropathy is identified as having autoantibodies specific for a FAT1 polypeptide as described herein, the mammal can be classified as having membranous nephropathy that includes the presence of those autoantibodies (e.g., membranous nephropathy that includes the presence of anti-FAT1 autoantibodies). In some cases, a mammal (e.g., a human) having membranous nephropathy that is identified as having autoantibodies specific for a FAT1 polypeptide as described herein can be classified as having membranous nephropathy that includes kidney tissue having an elevated level of FAT1 polypeptides.

Once a mammal (e.g., a human) having membranous nephropathy is identified as having kidney tissue (e.g., GBM) having an elevated level of a FAT1 polypeptide as described herein, the mammal can be classified as having membranous nephropathy that includes the presence of that kidney tissue (e.g., membranous nephropathy that includes the presence of kidney tissue such as GBM having an elevated level of FAT1 polypeptides). In some cases, a mammal (e.g., a human) having membranous nephropathy that is identified as having kidney tissue (e.g., GBM) having an elevated level of a FAT1 polypeptide as described herein can be classified as having membranous nephropathy that includes autoantibodies specific for a FAT1 polypeptide.

As described herein, this document also provides methods and materials for treating a mammal having membranous nephropathy. For example, a mammal (e.g., a human) having membranous nephropathy that is identified as having (a) autoantibodies specific for a FAT1 polypeptide and/or (b) kidney tissue (e.g., GBM) having an elevated level of a FAT1 polypeptide as described herein can be treated with one or more immunosuppressants. In some cases, a mammal (e.g., a human) having membranous nephropathy that is identified as having (a) autoantibodies specific for a FAT1 polypeptide and/or (b) kidney tissue (e.g., GBM) having an elevated level of a FAT1 polypeptide as described herein can be administered, or instructed to self-administer, one or more immunosuppressants to treat membranous nephropathy.

Any appropriate immunosuppressant can be administered to a mammal (e.g., a human that was identified as having (a) autoantibodies specific for a FAT1 polypeptide and/or (b) kidney tissue (e.g., GBM) having an elevated level of a FAT1 polypeptide as described herein) to treat membranous nephropathy. In some cases, an immunosuppressant used as described herein to treat membranous nephropathy can reduce inflammation and/or reduce B- cell autoantibody production within a mammal. Examples of immunosuppressants that can be used as described herein to treat membranous nephropathy include, without limitation, mycophenolate mofetil (e.g., Cellcept); steroids such as prednisone; B-cell inhibitors such as anti-CD20 antibodies (e.g., rituximab); calcineurin inhibitors such as cyclosporine and tacrolimus; and alkylating agents/chemotherapeutic drugs such as cyclophosphamide.

In some cases, two or more (e.g., two, three, four, five, six, or more) immunosuppressants can be administered to a mammal having membranous nephropathy (e.g., a human that was identified as having (a) autoantibodies specific for a FAT1 polypeptide and/or (b) kidney tissue (e.g., GBM) having an elevated level of a FAT1 polypeptide as described herein). For example, two immunosuppressants (e.g., prednisone and Cellcept) can be administered to a human having membranous nephropathy.

In some cases, one or more immunosuppressants can be administered to a mammal once or multiple times over a period of time ranging from days to months. In some cases, one or more immunosuppressive drugs can be given to achieve remission of membranous nephropathy, and then given during follow up periods to prevent relapse of the membranous nephropathy.

In some cases, one or more immunosuppressants can be formulated into a pharmaceutically acceptable composition for administration to a mammal (e.g., a human) having membranous nephropathy to reduce inflammation and/or to reduce B-cell autoantibody production within that mammal. For example, a therapeutically effective amount of an immunosuppressant can be formulated together with one or more pharmaceutically acceptable carriers (additives) and/or diluents. A pharmaceutical composition can be formulated for administration in solid or liquid form including, without limitation, in the form of sterile solutions, suspensions, sustained-release formulations, tablets, capsules, pills, powders, or granules.

Pharmaceutically acceptable carriers, fillers, and vehicles that can be used in a pharmaceutical composition described herein can include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

A pharmaceutical composition containing one or more immunosuppressants can be designed for oral or parenteral (including subcutaneous, intramuscular, intravenous, and intradermal) administration. When being administered orally, a pharmaceutical composition can be in the form of a pill, tablet, or capsule. Compositions suitable for parenteral administration can include aqueous and non-aqueous sterile injection solutions that can contain anti-oxidants, buffers, bacteriostats, and solutes that render the formulation isotonic with the blood of the intended recipient. The formulations can be presented in unit-dose or multi-dose containers, for example, sealed ampules and vials, and can be stored in a freeze dried (lyophilized) condition requiring the addition of the sterile liquid carrier, for example, water for injections, immediately prior to use. Extemporaneous injection solutions and suspensions can be prepared from sterile powders, granules, and tablets.

In some cases, a pharmaceutically acceptable composition including one or more immunosuppressants can be administered locally or systemically. For example, a composition provided herein can be administered locally by intravenous injection or blood infusion. In some cases, a composition provided herein can be administered systemically, orally, or by injection to a mammal (e.g., a human)

The effective amount (e.g., effective dose) of a composition containing one or more immunosuppressants can be any amount that reduces inflammation or B-cell autoantibody production (e.g., B-cell antibody production inhibition or reduction in the number of B-cells) within a mammal having membranous nephropathy without producing significant toxicity to the mammal. For example, an effective amount of rituximab to treat membranous nephropathy as described herein can be from about 500 mg to about 1.5 g (e.g., from about 500 mg to about 1.25 g, from about 500 mg to about 1.0 g, from about 500 mg to about 750 mg, from about 750 mg to about 1.5 g, from about 1 g to about 1.5 g, or from about 1.25 g to about 1.5 g) administered IV about two weeks apart. In some cases, an effective amount of rituximab to treat membranous nephropathy as described herein can be from about 200 mg/m² to about 500 mg/m² (e.g., from about 200 mg/m² to about 450 mg/m², from about 200 mg/m² to about 400 mg/m², from about 200 mg/m² to about 375 mg/m², from about 250 mg/m² to about 500 mg/m², from about 300 mg/m² to about 500 mg/m², from about 350 mg/m² to about 500 mg/m², or from about 350 mg/m² to about 400 mg/m²) administered weekly for about four weeks. If a particular mammal fails to respond to a particular amount, then the amount of an immunosuppressant can be increased by, for example, two fold. After receiving this higher amount, the mammal can be monitored for both responsiveness to the treatment and toxicity symptoms, and adjustments made accordingly. For example, levels of anti-FAT1 autoantibodies present within the mammal (e.g., within the blood of the mammal) can be monitored by an appropriate method (e.g., ELISA). In some cases, the effective amount of a composition containing one or more immunosuppressants can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition can require an increase or decrease in the actual effective amount administered.

The frequency of administration of one or more immunosuppressants can be any amount that reduces inflammation or B-cell autoantibody production (e.g., B-cell antibody production inhibition or reduction in the number of B-cells) within a mammal having membranous nephropathy without producing significant toxicity to the mammal. For example, the frequency of administration of an immunosuppressant can be from about once a day to about once a month (e.g., from about once a week to about once every other week). The frequency of administration of one or more immunosuppressants can remain constant or can be variable during the duration of treatment. A course of treatment with a composition containing one or more immunosuppressants can include rest periods. For example, a composition containing one or more immunosuppressants can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition may require an increase or decrease in administration frequency.

An effective duration for administering a composition containing one or more immunosuppressants can be any duration that reduces inflammation or B-cell autoantibody production (e.g., B-cell antibody production inhibition or reduction in the number of B-cells) within a mammal having membranous nephropathy without producing significant toxicity to the mammal. In some cases, the effective duration can vary from several days to several months. In general, the effective duration for administering a composition containing one or more immunosuppressants to treat membranous nephropathy can range in duration from about one month to about five years (e.g., from about two months to about five years, from about three months to about five years, from about six months to about five years, from about eight months to about five years, from about one year to about five years, from about one month to about four years, from about one month to about three years, from about one month to about two years, from about six months to about four years, from about six months to about three years, or from about six months to about two years). In some cases, the effective duration for administering a composition containing one or more immunosuppressants to treat membranous nephropathy can be for as long as the mammal is alive. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and/or the severity of one or more symptoms related to membranous nephropathy can be monitored. Any appropriate method can be used to determine whether or not membranous nephropathy is being treated. For example, immunological techniques (e.g., ELISA) can be performed to determine if the level of anti-FAT1 autoantibodies present within a mammal being treated as described herein is reduced following the administration of one or more immunosuppressants. Remission and relapse of the disease can be monitored by testing for one or more markers for membranous nephropathy. In some cases, remission can be ascertained by detecting the disappearance or reduction of autoantibodies having the ability to bind to a FAT1 polypeptide in the sera. In some cases, relapse of membranous nephropathy can be ascertained by a reappearance or elevation of autoantibodies to having the ability to bind to a FAT1 polypeptide in the sera.

The invention will be further described in the following examples, which do not limit the scope of the invention described in the claims.

### EXAMPLES

### Example 1: Hematopoietic Stem Cell Transplant-Membranous Nephropathy is Associated With Protocadherin FAT1

This Example identifies Protocadherin FAT1 (FAT1) as a target antigen in hematopoietic stem cell transplant (HSCT)-associated membranous nephropathy, and describes how FAT1 can be used as a serological biomarker and/or therapeutic target of HSCT-membranous nephropathy.

### Methods

### Patients and Sample Collection

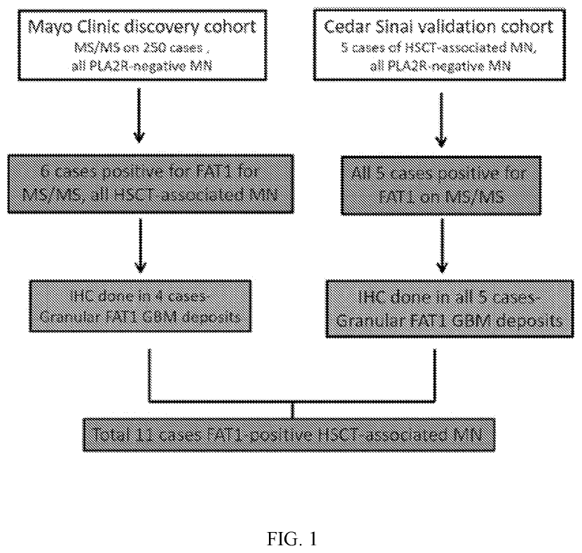

Biopsies received in the Renal Pathology Laboratory, Department of Laboratory Medicine and Pathology, Mayo Clinic, for diagnosis and interpretation were evaluated. The diagnosis of membranous nephropathy was confirmed by light microscopy, immunofluorescence microscopy including PLA2R studies, and electron microscopy. The clinical information was obtained from the accompanying charts. For detection of novel proteins, MS/MS was performed in 250 cases of PLA2R-negative cases that included the cases used for identification of an exostosin 1 (EXT1) polypeptide, an exostosin 2 (EXT2) polypeptide, a neural epidermal growth factor (EGF)-like 1 (NELL-1) polypeptide, a semaphorin-3B (SEMA3B) polypeptide, and a protocadherin-7 (PCDH7) polypeptide. 9 cases of FAT1-positive MN were detected by MS/MS (Mayo Clinic cohort, Case 1-5, 11-14) (FIG. 1). Case 11 was a recent case in which serum was available. All 9 cases were negative for spectral counts for THSD7A, EXT1/EXT2, NELL1, SEMA3B, and PCDH7 while baseline spectral counts for PLA2R were present in 3 of the 9 cases. Clinical information revealed that all 9 cases of FAT1-associated MN developed following allogeneic-HSCT.

For validation studies, MS/MS was performed in a validation cohort of eight cases of which five had history of HSCT and three were non-HSCT cases of PLA2R negative membranous nephropathy. All five cases of HSCT-associated membranous nephropathy showed spectral counts for FAT1 polypeptides (Cedar Sinai cohort, Case 6-10) (FIG. 1). The remaining three cases were negative for FAT1 polypeptides. The Mayo Clinic cohort kidney biopsy tissue was fixed in formalin, while the Cedar Sinai cohort tissue was fixed Bouin's fixative.

For control cases, MS/MS was performed on 116 cases that included 15 cases of time 0 kidney transplant biopsies, 17 cases of minimal change disease, 44 cases of focal segmental glomerulosclerosis, 7 cases of diabetic glomerulosclerosis, 5 cases of IgA nephropathy, and 28 cases of PLA2R-associated membranous nephropathy. The PLA2R-negative membranous nephropathy and control cases were the same cases that were used for MS/MS studies in the detection of EXT1/EXT2, NELL1, SEMA3B and PCDH7. None of the control cases showed any spectral counts for FAT1 polypeptides.

### Protein Identification by Laser Capture Microdissection, Trypsin Digestion, Nano-LC Orbitrap Tandem Mass Spectrometry (MS/MS)

For each case, 10 micron thick formalin-fixed paraffin sections (FFPE) were obtained and mounted on a special PEN membrane laser microdissection slide and using a Zeiss Palm Microbean microscope, the glomeruli were microdissected to reach approximately 250-550000 μM² per case. Resulting FFPE fragments were digested with trypsin and collected for MS/MS analysis. The trypsin digested polypeptides were identified by nano-flow liquid chromatography electrospray tandem MS/MS (nanoLC-ESI-MS/MS) using a Thermo Scientific Q-Exactive Mass Spectrometer (Thermo Fisher Scientific, Bremen, Germany) coupled to a Thermo Ultimate 3000 RSLCnano HPLC system. All MS/MS samples were analyzed using Mascot and X! Tandem set up to search a Swissprot human database. Scaffold (version 4.8.3, Proteome Software Inc., Portland, OR) was used to validate MS/MS based peptide and protein identifications. Polypeptides identifications were accepted at greater than 95.0% probability by the Scaffold Local FDR algorithm with polypeptides identifications requiring a two polypeptides minimum and a 95% probability using Protein Prophet.

The glomerular areas dissected for each FAT1 positive case was as follows: patient 1-116553 μM², patient 2-454701 μM², patient 3-277338 μM², patient 4-564474 μM², patient 5-1463913 μM², patient 6-51246 μM², patient 7-96333 μM², patient 8-126334 μM², patient 9-547169 μM², patient 10-610962 μM², patient 11-338810 μM², patient 12-100480 μM², patient 13-392136 μM², and patient 14-652180 μM².

### Immunohistochemical (IHC) Staining

IHC staining was performed using the Leica Bond RX stainer (Leica). FFPE tissues were sectioned at 5 microns and IHC staining was performed online. Slides for FAT1 polypeptide staining were pretreated for 5 minutes using Enzyme 2 (AR9551; Leica) and incubated in Protein Block (Dako) for 5 minutes. The anti-FAT1 polypeptide primary antibody (Rabbit Polyclonal; Abcam) was diluted to 1:200 in Background Reducing Diluent (Dako) and incubated for 15 minutes.

The detection system used was Polymer Refine Detection System (Leica). This system includes the hydrogen peroxidase block, post primary and polymer reagent, DAB, and hematoxylin. Immunostaining visualization was achieved by incubating slides for 10 minutes in DAB and DAB buffer (1:19 mixture) from the Bond Polymer Refine Detection System. To this point, slides were rinsed between steps with 1X Bond Wash Buffer (Leica). Slides were counterstained for five minutes using Schmidt hematoxylin and molecular biology grade water (1:1 mixture), followed by several rinses in 1X Bond wash buffer and distilled water. This was not the hematoxylin provided with the Refine kit. Once the immunochemistry process was completed, slides were removed from the stain and were rinsed in tap water for five minutes. Slides were dehydrated in increasing concentrations of ethyl alcohol and cleared in three changes of xylene prior to permanent coverslipping in xylene-based medium.

Confocal immunostaining: Immunofluorescence staining was performed on 8 μm sections cut from formalin (Case 5) and Bouin's (Case 8)-fixed biopsy samples. Sections from two cases of PLA2R-associated membranous nephropathy were also stained. Slides were deparaffinized and antigen retrieval was performed with Proteinase-K treatment for 5 minutes at room temperature. Following quenching in 100 mM NH₄Cl for 20 minutes and blocking (5% normal donkey serum, 5% BSA in 0.1% Triton X-100, PBS) for 2 hours, slides were incubated with primary antibody at 4° C. overnight. The following day, the secondary antibody incubation was performed at room temperature for 2 hours. Washes using PBS+0.05% Triton X were performed between the incubations. Primary antibodies against FAT1 polypeptides (catalog no. ab198892, Abcam) and secondary antibodies Donkey anti-rabbit AF555N (Thermo Scientific) were employed. Microscope images (Zeiss LSM780) were acquired using a 20× lens (NA 0.3), and images were prepared using Photoshop. All exposure levels were identical within the groups.

### Elution of IgG From Kidney Biopsy Specimens

IgG was acid eluted from frozen kidney biopsy specimens. Serial cryostat sections of 4 μm thickness were mounted side by side on a glass slide. The slides were thawed, fixed with pre-chilled 100% acetone for 10 minutes at room temperature, and then washed for 5 minutes with phosphate-buffered saline (0.01 M, phosphate, pH 7.2). The slide sections were covered with 0.2 mL of a 0.02 M citrate buffer (pH 3.2) and incubated overnight in a humid chamber at 4° C. The eluate was extracted with a calibrated syringe and neutralized with 0.4 M NaOH to a pH of 7.2. The eluate containing anti-FAT1 IgG was obtained from four patients with FAT1-associated membranous nephropathy that were pooled together. The eluate containing anti-PLA2R IgG was obtained from six patients with PLA2R-associated membranous nephropathy.

### Western Blot Analysis

A recombinant protein corresponding to antigenic determinants in human FAT1 polypeptides (Novus Biologicals) was used under non-reducing and reducing conditions. The target molecular weight and dominant band is expected at 33 kDa. The polypeptide (400 ng) was diluted with non-reduced and reduced Laemmli sample buffer (BioRad) and boiled for 5 minutes. The samples were loaded into Criterion 4%-15% TGX gels (BioRad) and electrophoresed in Tris-glycine-SDS running buffer. Polypeptides were transferred to nitrocellulose membrane (0.20 μm pore size) according to standard protocols. Membranes were incubated overnight at 4° C. with rabbit anti-human FAT1 antibodies (0.2 μg/mL, recommended dilution range 0.04-0.4 μg/mL, Novus Biologicals), eluates from FAT1-associated membranous nephropathy and PLA2R-associated membranous nephropathy, and patient serum from FAT1-associated membranous nephropathy (1:50 and 1:100 dilutions, patient 11). Serum from patients with PLA2R-associated membranous nephropathy, PLA2R-negative membranous nephropathy, IgA nephropathy, myeloperoxidase (MPO) ANCA-associated vasculitis, and one healthy control was probed against reduced FAT1 polypeptides as negative controls. Subsequently, blots were washed and incubated 1 hour at room temperature with goat anti-rabbit and goat anti-human IgG Fc (1:15000 and 1:5000, respectively, LI-COR®) and with mouse anti-human IgG4 Fc HRP (1:500, Thermo Fischer Scientific). Anti-human IgG4 antibodies were used for detection of anti-PLA2R IgG since anti-PLA2R antibodies belong to IgG4 subclass. Near infrared fluorescence was detected at the 700 and 800 nm channel and by chemiluminescence in the Odyssey Infrared Imaging System (LI-COR® Biosciences, Lincoln, NE).

### Results

Mayo Clinic discovery cohort (9 cases, patients 1-5, 11-14): A total of 1487 allogeneic-HSCT were done at Mayo Clinic. Nine (0.6%) patients developed MN. Of the 9 cases, tissue was available for typing by MS/MS and IHC in 6 cases. One (16.7%) patient was positive for PLA2R. The remaining 5 (83.3%) patients were positive for FAT1-associated MN.

An additional 4 cases of HSCT-associated MN were from other hospitals. These cases were initially diagnosed as PLA2R-negative MN. MS/MS showed that these cases were positive for FAT1, bringing the total of the discovery (Mayo Clinic) cohort to 9.

Cedars Sinai validation cohort (5 cases, patients 6-10): This cohort included 5 cases of HSCT-associated MN. All 5 (100%) cases were positive for FAT1 on MS/MS and IHC. The Cedar Sinai cohort included all HSCT-associated MN cases received in the renal biopsy service dating to 2006.

All 14 cases developed MN following allogeneic HSCT. None of the MN cases developed following autologous HSCT. The consolidated results are described below.

### Laser Dissection and MS/MS Detection of FAT1 in PLA2R-Negative Membranous Nephropathy Biopsies

A unique polypeptide, FAT1, was detected by MS/MS in the glomeruli of 14 cases of MN (FIG. 2). The counts ranged from 8 to 39 with an average total spectral count of 20.9 (SD±10.1). The average spectral counts of FAT1 were lower than PLA2R (86.1, S.D±27.5), EXT1/EXT2 (EXT1 65.3, S.D±34.6, EXT2 83.4, S.D±38.4), and NELL-1 (63.1, S.D±21.6) in PLA2R-, EXT1/EXT2-, and NELL1-associated MN, respectively. On the other hand, the counts were comparable to SEMA3B (23.7±16.5) and higher than PCDH7 (13.2±6.6). In addition, the finding of FAT1 was unique in this subset of PLA2R-negative MN and importantly, all control cases including 15-time 0 transplant biopsies, 73 other glomerulopathies, and 28 PLA2R-positive MN cases were negative for FAT1.

The spectral counts of all 11 FAT1-positive cases along with a representative sequence coverage map of PCDH7 are shown in FIG. 2A-B. The MS/MS spectra match from one case is shown in FIG. 2C. None of the FAT1 polypeptide positive cases show any spectral counts for EXT1/EXT2, THSD7A, NELL1, SEMA3B or PCDH7 while baseline PLA2R counts were detected in four of the 11 cases, but the counts were much lower than the FAT1 polypeptide counts and appear similar to the baseline PLA2R counts seen in EXT1/EXT2, NELL1 and SEMA3B-associated membranous nephropathy.

All four sub classes of IgG were detected in FAT1-associated membranous nephropathy, with average spectral counts of IgG1 25.0 (S.D±8.8), IgG2 17.1 (S.D±10.8), IgG3 16.9 (S.D±8.5), and IgG4 13.5 (S.D±6.9).

### Immunohistochemical and Confocal Staining for FAT1 in PLA2R-Negative Membranous Nephropathy Biopsies

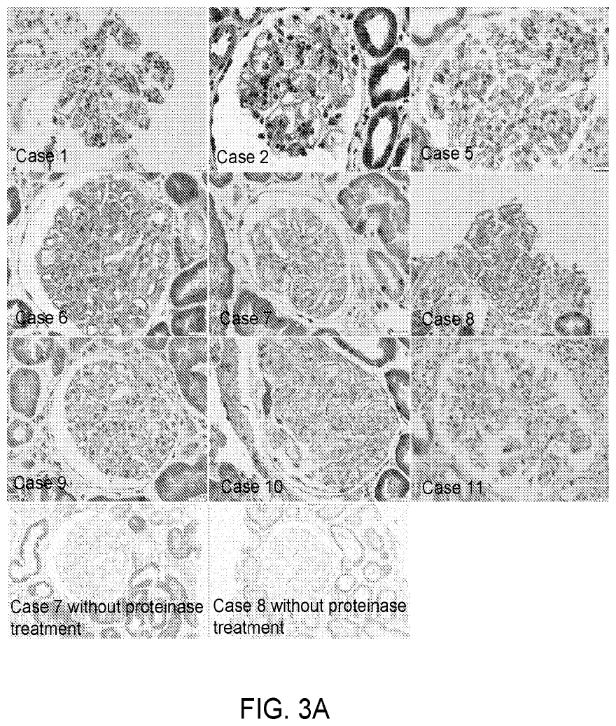

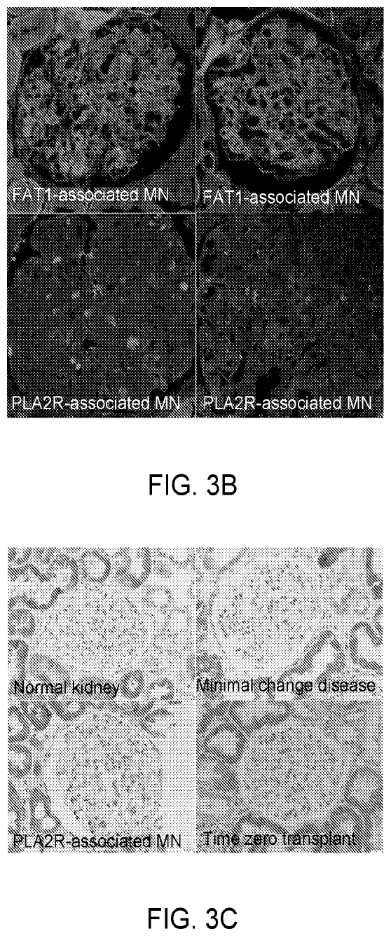

Fourteen cases were positive for FAT1 by MS/MS. Tissue was available in 12 of the 14 cases for immunohistochemical staining. All 12 cases showed granular (1-3+/3) staining for FAT1 along the GBM (FIG. 3A). The positive FAT1 granular staining mirrored the granular IgG along the GBM seen on IF in each case. Case 13 was very early MN with mild IgG on IF and only few subepithelial deposits on EM (stage1) and showed only few loops with mild granular staining for FAT1. Also, FAT1 staining along the GBM was only seen following protease digestion, indicating FAT1 epitopes recognized by the commercial anti-FAT1 antibody were likely masked. FAT1 staining without protease digestion was negative in FAT1 positive cases (bottom left panel, FIG. 3A). Bowman's capsule and mesangial staining was negative. Tubular basement membrane staining was negative. The tubular epithelial cell cytoplasm was strongly positive for FAT1, the staining is likely non-specific. Confocal microscopy studies also showed that glomeruli are positive for FAT1 along the capillary walls in 2 cases of HSCT-associated MN (one each from discovery cohort and validation cohort) and negative for FAT1 in 2 cases of PLA2R-associated MN (FIG. 3B).

Control cases were negative for FAT1 polypeptides staining along the GBM. Negative staining for FAT1 polypeptides in a representative case of normal kidney (nephrectomy specimen), minimal change disease, PLA2R-associated membranous nephropathy and time 0 kidney transplant are shown in FIG. 3C. There was also no staining in cases of focal segmental glomerulosclerosis (FSGS) and lupus nephritis (not shown).

### IgG Elution and Western Blot Studies

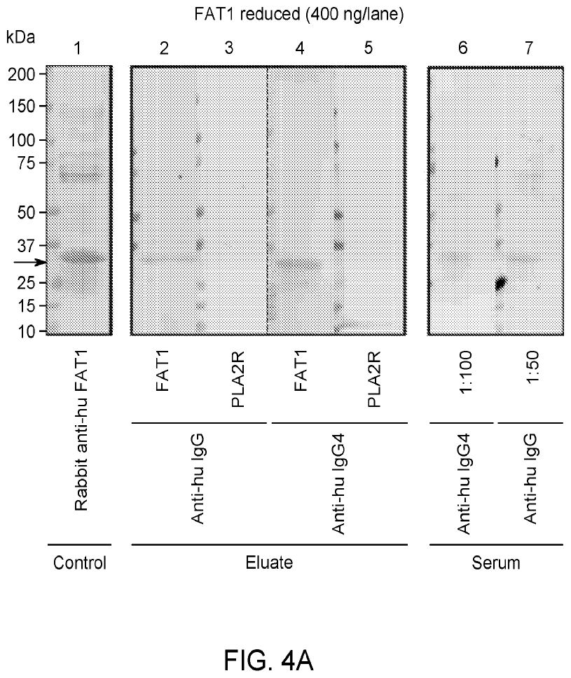

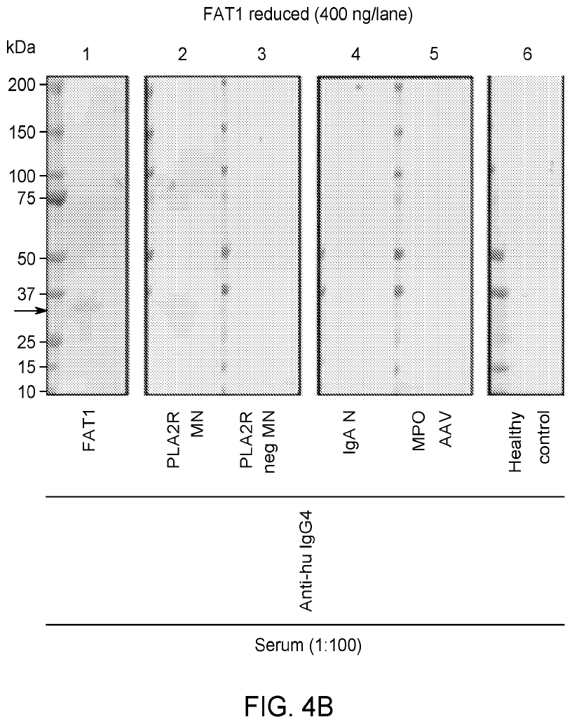

Western blot analyses were performed using recombinant human FAT1 to determine the presence of anti-FAT1 antibodies in the eluate obtained from pooled kidney biopsies and serum from a recent FAT1-associated MN case (patient 11) (FIG. 4). A recombinant 33 kDa human FAT1 protein corresponding to antigenic determinants (Novus Biologicals©) was used in western blot analysis. FAT1 was detected by rabbit anti-human FAT1 under both non-reducing (FIG. 4A) and reducing (FIG. 4B) conditions with a dominant band as expected at approximately 33 kDa (FIGS. 4A and 4B, lane 1). The same band was detected after exposure of the recombinant human FAT1 to IgG obtained from the eluate of FAT1-associated MN using anti-human IgG (FIGS. 4A and 4B, lane 2). More specifically, the band was seen after exposure of recombinant human FAT1 to eluate from FAT1-associated MN using anti-human IgG4 (FIG. 4A, lane 3, and 4B, lane 4) but not after exposure to the eluate from PLA2R-associated MN (FIG. 4A, lane 4 and 4B lane 3 and 5). Finally, the same band was seen using serum from patient 11 using anti-human IgG4 (FIG. 4B, lane 6) and anti-human IgG (FIG. 4B, lane 7). Anti-FAT1 antibodies were not detected in serum from patients with PLA2R-associated-MN, PLA2R negative MN, IgA nephropathy, MPO-AAV or in serum from a healthy control (FIG. 4C).

### Clinical and Kidney Biopsy Findings of FAT1-Associated Membranous Nephropathy

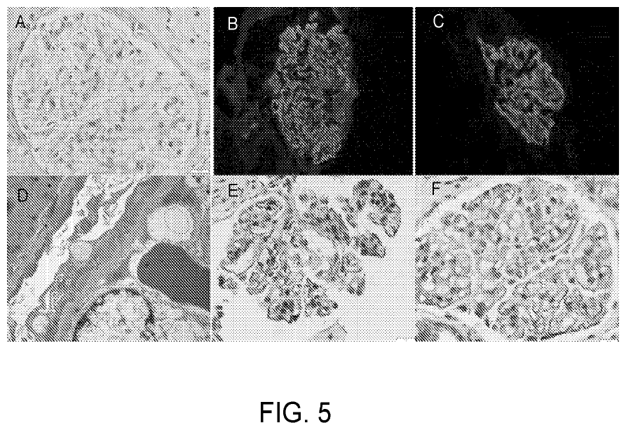

The mean age of FAT1-associated MN was 60.3±8.6 years (Table 1A). MN occurred 2.4±0.8 years after HSCT. There were 9 males and 5 females. The mean serum creatinine and proteinuria at kidney biopsy was 1.4±0.5 mg/dL and 7.8±6.0 gms/day, respectively. HSCT was done for treatment of 8 patients with acute myelogenous leukemia, 2 patients with myelodysplastic syndrome, and 1 patient each with chronic lymphocytic leukemia, essential thrombocytopenia, myelofibrosis and lymphoplasmacytic disease. Kidney biopsy findings showed granular IgG (2-3+), kappa (1-3+) and lambda light chains (1-3+) in all cases (Table 1B). Subtyping of IgG done in 6 cases with available tissue showed dominant IgG4 (3+) in all cases, in 1 case there was also IgG2 (2+) present. There was only mild C3 present (0-1+). Other immunoglobulins and C1q were absent. 3 cases also showed tubular basement membrane deposits that were positive for IgG. Mass spectrometry of the tubular deposits was negative for FAT1. Chronic changes in 12 of the 14 cases were mild including focal global glomerulosclerosis (median 10%) and tubular atrophy and interstitial fibrosis (median 10-20%). In 2 of the 10 cases there was extensive (>50%) tubular atrophy and interstitial fibrosis present. A representative case with kidney biopsy findings (patient 1) is shown in FIG. 5. Treatment details were available for the 5 Mayo Clinic patients. Patient 2 was treated with prednisone and rituximab and achieved partial remission. She died 3 months after diagnosis of MN due to congestive heart failure. Patient 3 was treated with rituximab and achieved complete remission. Patient 12 achieved complete remission with prednisone and cyclosporine but died 13 years later of probable sepsis. Patient 13 achieved complete remission with prednisone. Patient 14 achieved partial remission with prednisone and cyclosporine but died 4 months from sepsis. Overall, follow-up of the 11 cases showed complete remission in 6 patients (proteinuria of <0.3 grams/24 hours), 3 patients continued to have significant proteinuria (nephrotic range in 1), 1 patient was a recent case, and 6 patients died (Table 1). Data available in 4 of 6 patients that died showed cause of death as congestive heart failure in one patient and sepsis as the cause of death in 3 cases.

Table 1. Clinical and pathologic findings in FAT1-associated Membranous Nephropathy (Mayo Clinic cohort patients 1-5, Cedar Sinai cohort patients 6-10).

TABLE 1A
Clinical characteristics.

| | | | | | Years | Serum | Proteinuria | | Serum | Proteinuria |
|---|---|---|---|---|---|---|---|---|---|---|
| Case | | | | Primary | to | creatinine | (grams/24 | Outcome | creatinine | (grams/24 |
| number | Age | Sex | Transplant | disease | MN | (mg/dL) | hours) | (months) | (mg/dL) | hours) |
| 1 | 57 | F | HSCT | AML | 1.5 | NA | 3 | Deceased | — | — |
| 2 | 69 | F | HSCT | AMI | 4 | 1.2 | 3.8 | Deceased | — | — |
| 3 | 69 | F | HSCT | AML | 2 | 2 | 3.5 | 24 | 1.7 | 0.3 (CR) |
| 4 | 54 | F | HSCT | AML | 3 | 2.6 | 17 | 64 | 1.0 | 0.2 (CR) |
| 5 | 54 | M | HSCT | CLŁ | — | 1.4 | 22 | Deceased | — | — |
| 6 | 53 | M | HSCT | MDS | 3 | 1.3 | 4.4 | 32 | 1.5 | 0.4 (CR) |
| 7 | 63 | M | HSCT | AML | 2 | 1 | 7 | N/A | N/A | N/A |
| 8 | 42 | F | HSCT | Essential | 2 | 1.1 | 15 | 65 | 1.7 | 0.3(CR) |
| | | | | thrombocytopenia | | | | | | |
| 9 | 71 | M | HSCT | Myelofibrosis | 2 | 1.2 | 8 | Deceased | — | — |
| 10 | 68 | M | HSCT | AML | 2 | 1.1 | 5.6 | N/A | N/A | N/A |
| 11 | 69 | M | HSCT | AML | 4 | 1.5 | 9 | Recent | — | — |
| | | | | | | | | case | | |

TABLE 1B
Pathologic findings.

| | | | Interstitial | | | |
|---|---|---|---|---|---|---|
| | | Number of | fibrosis and | | | |
| Case | Number of | sclerosed | tubular | Immunofluorescence | TBM | EM |
| number | glomeruli | glomeruli | atrophy (%) | microscopy | deposits | stage |
| 1 | 17 | 2 | 20 | IgG 3+, C3 1+, IgG4 3+ | No | I |
| 2 | 17 | 6 | 20 | IgG 3+, C3 1+ | Yes | II |
| 3 | 43 | 18 | 60 | IgG 3+, C3 1+, IgG4 3+ | Yes | II |
| 4 | 86 | 68 | 80 | IgG 3+, C3 1+ | Yes | II |
| 5 | 36 | 4 | 20 | IgG 3+ | No | II |
| 6 | 6 | 1 | 20 | IgG 3+, C3 1+ | No | II |
| 7 | 57 | 2 | 5 | IgG 3+, C3 1+, IgG4 3+, IgG2 2+ | No | II |
| 8 | 50 | 9 | 5 | IgG 2+, C3 1+ | No | II |
| 9 | 55 | 2 | 10 | IgG 2+, IgG4 2+ | No | II |
| 10 | 41 | 2 | 10 | IgG 3+, C3 1+, IgG4 3+ | No | II |
| 11 | 18 | 1 | 10 | IgG 2+, C3 1+, IgG4 3+ | No | II |

Together, these results demonstrate that the FAT1 polypeptide is the target antigen in a HSCT-associated membranous nephropathy. Accordingly, the presence of (a) autoantibodies specific for a FAT1 polypeptide and/or (b) a GBM having an elevated level of a FAT1 polypeptide can be used to identify a mammal (e.g., a human that has undergone HSCT) as having a FAT1-positive membranous nephropathy. In some cases, a mammal (e.g., a human that has undergone HSCT) identified as having a FAT1-positive membranous nephropathy can be treated by administering one or more immunosuppressive agents to the mammal.

### Example 2: Identifying FAT1 Polypeptide Positive Membranous Nephropathy

A blood sample (e.g., serum) is obtained from a human having membranous nephropathy. The obtained sample is examined for the presence of autoantibodies specific for a FAT1 polypeptide.

If autoantibodies specific for a FAT1 polypeptide are detected in the sample, as compared to a control level, then the human is classified as having a FAT1 positive membranous nephropathy.

### Example 3: Treating FAT1 Positive Membranous Nephropathy

A human identified as having autoantibodies specific for a FAT1 polypeptide is administered one or more immunosuppressive agents (e.g., corticosteroids, cyclosporine, or a B-cell reduction or depletion agent such as Rituximab)

The administered immunosuppressive agent(s) can reduce inflammation and/or B-cell autoantibody production.

The administered immunosuppressive agent(s) can reduce the level of autoantibodies specific for a FAT1 polypeptide present within the human.

### Example 4: Identifying FAT1 Positive Membranous Nephropathy

A kidney tissue sample is obtained from a human having membranous nephropathy. The obtained sample is examined for an elevated level of a FAT1 polypeptide.

If an elevated level of a FAT1 polypeptide is detected in the sample, as compared to a control level, then the human is classified as having a FAT1 positive membranous nephropathy.

### Example 5: Treating FAT1 Positive Membranous Nephropathy

A human identified as having an elevated level of a FAT1 polypeptide in the GBM of the kidney is administered one or more immunosuppressive agents (e.g., corticosteroids, cyclosporine, or a B-cell reduction or depletion agent such as Rituximab).

The administered immunosuppressive agent(s) can reduce inflammation and/or B-cell autoantibody production.

The administered immunosuppressive agent(s) can reduce a level of autoantibodies specific for a FAT1 polypeptide present within the human.

### Example 6: Exemplary Embodiments

Embodiment 1. A method for identifying a mammal comprising an hematopoietic stem cell transplant as having membranous nephropathy comprising an elevated level of a protocadherin FAT1 (FAT1) polypeptide within kidney tissue of said mammal, wherein said method comprises:
(a) determining the presence or absence of autoantibodies specific for said FAT1 polypeptide within a sample obtained from said mammal,
(b) classifying said mammal as having said membranous nephropathy if said autoantibodies are present within said mammal, and
(c) classifying said mammal as not having said membranous nephropathy if said autoantibodies are absent within said mammal.

Embodiment 2. The method of embodiment 1, wherein said mammal is a human.

Embodiment 3. The method of any one of embodiments 1-2, wherein said sample is a blood sample.

Embodiment 4. The method of any one of embodiments 1-3, wherein said membranous nephropathy lacks an elevated level of a protocadherin-7 (PCDH7) polypeptide, a semaphorin-3B (SEMA3B) polypeptide, a neural epidermal growth factor (EGF)-like 1 (NELL-1) polypeptide, an exostosin 1 (EXT1) polypeptide, an exostosin 2 (EXT2) polypeptide, a PLA2R polypeptide, or a THSD7A polypeptide within said kidney tissue.

Embodiment 5. The method of any one of embodiments 1-3, wherein said membranous nephropathy lacks an elevated level of a PCDH7 polypeptide, a SEMA3B polypeptide, a NELL-1 polypeptide, an EXT1 polypeptide, an EXT2 polypeptide, a PLA2R polypeptide, and a THSD7A polypeptide within said kidney tissue.

Embodiment 6. The method of any one of embodiments 1-5, wherein said method comprises detecting the presence of said autoantibodies and classifying said mammal as having said membranous nephropathy.

Embodiment 7. The method of any one of embodiments 1-5, wherein said method comprises detecting the absence of said autoantibodies and classifying said mammal as not having said membranous nephropathy.

Embodiment 8. A method for identifying a mammal comprising an hematopoietic stem cell transplant as having membranous nephropathy having kidney tissue comprising an elevated level of a FAT1 polypeptide, wherein said method comprises:
(a) determining the presence or absence of said kidney tissue within a sample obtained from said mammal,
(b) classifying said mammal as having said membranous nephropathy if said presence is determined, and
(c) classifying said mammal as not having said membranous nephropathy if said absence is determined.

Embodiment 9. The method of embodiment 8, wherein said mammal is a human.

Embodiment 10. The method of any one of embodiments 8-9, wherein said kidney tissue lacks an elevated level of a PCDH7 polypeptide, a SEMA3B polypeptide, a NELL-1 polypeptide, an EXT1 polypeptide, an EXT2 polypeptide, a PLA2R polypeptide, or a THSD7A polypeptide.

Embodiment 11. The method of any one of embodiments 8-9, wherein said kidney tissue lacks an elevated level of a PCDH7 polypeptide, a SEMA3B polypeptide, a NELL-1 polypeptide, an EXT1 polypeptide, an EXT2 polypeptide, a PLA2R polypeptide, and a THSD7A polypeptide.

Embodiment 12. The method of any one of embodiments 8-11, wherein said method comprises detecting said presence and classifying said mammal as having said membranous nephropathy.

Embodiment 13. The method of any one of embodiments 8-11, wherein said method comprises detecting said absence and classifying said mammal as not having said membranous nephropathy.

Embodiment 14. A method for identifying a mammal comprising an hematopoietic stem cell transplant as having membranous nephropathy having autoantibodies specific for a FAT1 polypeptide, wherein said method comprises:
(a) determining the presence or absence of said autoantibodies within a sample obtained from said mammal,
(b) classifying said mammal as having said membranous nephropathy if said autoantibodies are present within said mammal, and
(c) classifying said mammal as not having said membranous nephropathy if said autoantibodies are absent within said mammal.

Embodiment 15. The method of embodiment 14, wherein said mammal is a human.

Embodiment 16. The method of any one of embodiments 14-15, wherein said sample is a blood sample.

Embodiment 17. The method of any one of embodiments 14-16, wherein kidney tissue of said mammal lacks an elevated level of a PCDH7 polypeptide, a SEMA3B polypeptide, a NELL-1 polypeptide, an EXT1 polypeptide, an EXT2 polypeptide, a PLA2R polypeptide, or a THSD7A polypeptide.

Embodiment 18. The method of any one of embodiments 14-16, wherein kidney tissue of said mammal lacks an elevated level of a PCDH7 polypeptide, a SEMA3B polypeptide, a NELL-1 polypeptide, an EXT1 polypeptide, an EXT2 polypeptide, a PLA2R polypeptide, and a THSD7A polypeptide.

Embodiment 19. The method of any one of embodiments 14-18, wherein said method comprises detecting said presence and classifying said mammal as having said membranous nephropathy.

Embodiment 20. The method of any one of embodiments 14-18, wherein said method comprises detecting said absence and classifying said mammal as not having said membranous nephropathy.

Embodiment 21. A method for treating a mammal having membranous nephropathy, wherein said method comprises:
(a) identifying a mammal as having membranous nephropathy comprising (i) autoantibodies specific for a FAT1 polypeptide or (ii) kidney tissue comprising an elevated level of said FAT1 polypeptide, and
(b) administering an immunosuppressant to said mammal.

Embodiment 22. The method of embodiment 21, wherein said mammal is a human.

Embodiment 23. The method of any one of embodiments 21-22, wherein said mammal is identified as having said membranous nephropathy comprising said autoantibodies.

Embodiment 24. The method of any one of embodiments 21-23, wherein said mammal is identified as having said membranous nephropathy comprising said kidney tissue.

Embodiment 25. The method of any one of embodiments 21-24, wherein said immunosuppressant is a B-cell inhibitor.

Embodiment 26. The method of embodiment 25, wherein said B-cell inhibitor is rituximab.

Embodiment 27. The method of any one of embodiments 21-24, wherein said immunosuppressant is a calcineurin inhibitor.

Embodiment 28. The method of embodiment 27, wherein said calcineurin inhibitor is cyclosporine or tacrolimus.

Embodiment 29. The method of any one of embodiments 21-24, wherein said immunosuppressant is an mTOR inhibitor.

Embodiment 30. The method of embodiment 29, wherein said mTOR inhibitor is sirolimus or everolimus.

Embodiment 31. The method of any one of embodiments 21-24, wherein said immunosuppressant is a DNA damage inducer.

Embodiment 32. The method of embodiment 31, wherein said DNA damage inducer is chlorambucil.

Embodiment 33. The method of any one of embodiments 21-32, wherein the level of autoantibodies present within said mammal is reduced by at least 5 percent following said administering step.

Embodiment 34. The method of any one of embodiments 21-32, wherein the level of autoantibodies present within said mammal is reduced by at least 25 percent following said administering step.

Embodiment 35. The method of any one of embodiments 21-32, wherein the level of autoantibodies present within said mammal is reduced by at least 50 percent following said administering step.

Embodiment 36. A method for treating a mammal having membranous nephropathy, wherein said method comprises administering an immunosuppressant to a mammal identified as having membranous nephropathy comprising (i) autoantibodies specific for a FAT1 polypeptide or (ii) kidney tissue comprising an elevated level of said FAT1 polypeptide.

Embodiment 37. The method of embodiment 36, wherein said mammal is a human.

Embodiment 38. The method of any one of embodiments 36-37, wherein said mammal was identified as having said membranous nephropathy comprising said autoantibodies.

Embodiment 39. The method of any one of embodiments 36-37, wherein said mammal was identified as having said membranous nephropathy comprising said kidney tissue.

Embodiment 40. The method of any one of embodiments 36-39, wherein said immunosuppressant is a B-cell inhibitor.

Embodiment 41. The method of embodiment 40, wherein said B-cell inhibitor is rituximab.

Embodiment 42. The method of any one of embodiments 36-39, wherein said immunosuppressant is a calcineurin inhibitor.

Embodiment 43. The method of embodiment 42, wherein said calcineurin inhibitor is cyclosporine or tacrolimus.

Embodiment 44. The method of any one of embodiments 36-39, wherein said immunosuppressant is an mTOR inhibitor.

Embodiment 45. The method of embodiment 44, wherein said mTOR inhibitor is sirolimus or everolimus.

Embodiment 46. The method of any one of embodiments 36-39, wherein said immunosuppressant is a DNA damage inducer.

Embodiment 47. The method of embodiment 46, wherein said DNA damage inducer is chlorambucil.

Embodiment 48. The method of any one of embodiments 36-47, wherein the level of autoantibodies present within said mammal is reduced by at least 5 percent following said administering step.

Embodiment 49. The method of any one of embodiments 36-47, wherein the level of autoantibodies present within said mammal is reduced by at least 25 percent following said administering step.

Embodiment 50. The method of any one of embodiments 36-47, wherein the level of autoantibodies present within said mammal is reduced by at least 50 percent following said administering step.

Embodiment 51. A method for treating a mammal having membranous nephropathy and kidney tissue comprising an elevated level of a FAT1 polypeptide, wherein said method comprises administering an immunosuppressant to said mammal.

Embodiment 52. The method of embodiment 51, wherein said mammal is a human.

Embodiment 53. The method of any one of embodiments 51-52, wherein said mammal comprises autoantibodies specific for said polypeptide.

Embodiment 54. The method of any one of embodiments 51-52, wherein said mammal was identified as having said kidney tissue.

Embodiment 55. The method of any one of embodiments 51-54, wherein said immunosuppressant is a B-cell inhibitor.

Embodiment 56. The method of embodiment 55, wherein said B-cell inhibitor is rituximab.

Embodiment 57. The method of any one of embodiments 51-54, wherein said immunosuppressant is a calcineurin inhibitor.

Embodiment 58. The method of embodiment 57, wherein said calcineurin inhibitor is cyclosporine or tacrolimus.

Embodiment 59. The method of any one of embodiments 51-54, wherein said immunosuppressant is an mTOR inhibitor.

Embodiment 60. The method of embodiment 59, wherein said mTOR inhibitor is sirolimus or everolimus.

Embodiment 61. The method of any one of embodiments 51-54, wherein said immunosuppressant is a DNA damage inducer.

Embodiment 62. The method of embodiment 61, wherein said DNA damage inducer is chlorambucil.

Embodiment 63. The method of any one of embodiments 51-62, wherein the level of autoantibodies present within said mammal is reduced by at least 5 percent following said administering step.

Embodiment 64. The method of any one of embodiments 51-62, wherein the level of autoantibodies present within said mammal is reduced by at least 25 percent following said administering step.

Embodiment 65. The method of any one of embodiments 51-62, wherein the level of autoantibodies present within said mammal is reduced by at least 50 percent following said administering step.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-20. (canceled)

21. A method for treating a mammal having membranous nephropathy, wherein said method comprises:
(a) identifying a mammal as having membranous nephropathy comprising (i) autoantibodies specific for a FAT1 polypeptide or (ii) kidney tissue comprising an elevated level of said FAT1 polypeptide, and
(b) administering an immunosuppressant to said mammal.

22. The method of claim 21, wherein said mammal is a human.

23. The method of claim 21, wherein said mammal is identified as having said membranous nephropathy comprising said autoantibodies.

24. The method of claim 21, wherein said mammal is identified as having said membranous nephropathy comprising said kidney tissue.

25. The method of claim 21, wherein said immunosuppressant is a B-cell inhibitor.

26. The method of claim 25, wherein said B-cell inhibitor is rituximab.

27. The method of claim 21, wherein said immunosuppressant is a calcineurin inhibitor.

28. The method of claim 27, wherein said calcineurin inhibitor is cyclosporine or tacrolimus.

29. The method of claim 21, wherein said immunosuppressant is an mTOR inhibitor.

30. The method of claim 29, wherein said mTOR inhibitor is sirolimus or everolimus.

31. The method of claim 21, wherein said immunosuppressant is a DNA damage inducer.

32. The method of claim 31, wherein said DNA damage inducer is chlorambucil.

33. The method of claim 21, wherein the level of autoantibodies present within said mammal is reduced by at least 5 percent following said administering step.

34. A method for treating a mammal having membranous nephropathy, wherein said method comprises administering an immunosuppressant to a mammal identified as having membranous nephropathy comprising (i) autoantibodies specific for a FAT1 polypeptide or (ii) kidney tissue comprising an elevated level of said FAT1 polypeptide.

35. A method for treating a mammal having membranous nephropathy and kidney tissue comprising an elevated level of a FAT1 polypeptide, wherein said method comprises administering an immunosuppressant to said mammal.
